(19) United States
(12) Patent Application Publication
(10) Pub. No.: US 2013/0059008 A1
(43) Pub. Date: Mar. 7, 2013

(54) DRYING METHODS FOR TUNING MICROPARTICLE PROPERTIES

(71) Applicants: Jeffrey L. Atkinson, Helena, AL (US); Brian Keith Chambers, Pelham, AL (US); Michael E. Hudson, Hayden, AL (US)

(72) Inventors: Jeffrey L. Atkinson, Helena, AL (US); Brian Keith Chambers, Pelham, AL (US); Michael E. Hudson, Hayden, AL (US)

(21) Appl. No.: 12/692,029

(22) Filed: Jan. 22, 2010

Related U.S. Application Data
(60) Provisional application No. 61/146,862, filed on Jan. 23, 2009.

(51) Int. Cl. A61K 38/16; A61P 43/00

(52) U.S. Cl. 424/489; 514/1.1

(57) ABSTRACT

Described herein are drying methods for tuning one or more properties of a microparticle. In one aspect, the release profile of a microparticle comprising a bioactive agent therein can be affected by the disclosed drying methods.

### CROSS-REFERENCE TO RELATED APPLICATIONS

This application is based upon and claims the benefit of priority from prior U.S. Provisional Application No. 61/146,862, filed Jan. 23, 2009, which is incorporated herein by reference.

### BACKGROUND

In order for a bioactive agent to work effectively, it must be delivered to a subject in a way that is both safe and effective. An ideal pharmacokinetic profile of a bioactive agent is one which allows for therapeutic concentrations of the bioactive agent to be reached in a subject, while not exceeding the maximum tolerable dose. For certain pharmacological applications, concentrations of the bioactive agent should remain at a therapeutic level for an extended period of time until the desired therapeutic result is achieved.

Unfortunately, conventional routes for administering bioactive agents often do not provide ideal pharmacokinetic profiles, especially for bioactive agents that display high toxicity and/or narrow therapeutic windows. It is known in the art that one way of affecting a pharmocokinetic profile of a bioactive agent is to encapsulate the bioactive agent in a microparticle. The microparticle can degrade over time, thereby releasing the bioactive agent according to a release profile that is influenced by the microparticle. Oftentimes, however, the microparticle may still not provide for a desired release profile, and in some instances can even result in an undesirable release profile.

As such, a need exists for microparticles and methods for the manufacture thereof that can substantially provide a suitable release profile for a bioactive agent contained in or on the microparticle. These needs and other needs are satisfied by the present invention.

### SUMMARY

Described herein are methods for preparing a microparticle having a selected release profile for the release of a bioactive agent contained therein. The disclosed methods can, in one aspect, allow for the tuning of one or more microparticle properties, depending on the method parameters, such as the drying parameters. In one aspect, a desired release profile of a bioactive agent can be affected or provided by adjusting or tuning the microparticle preparation method parameters disclosed herein.

In one aspect, a method for preparing a microparticle having a selected release profile for the release of a bioactive agent contained therein comprises: a) providing a slurry comprising a microparticle having a releasable bioactive agent therein; b) selecting a release profile for the bioactive agent; and c) drying the microparticle under a set of drying parameters such that the selected release profile is substantially achieved.

In a further aspect, a method for drying a microparticle comprises: a) providing a slurry comprising a microparticle having a releasable bioactive agent therein; and b) drying the microparticle using an agitated filter dryer or a stirred cell filter dryer under a set of selected drying parameters.

Also disclosed are microparticles made by the disclosed methods.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 is a plot of particle size distribution of BSA-loaded microparticles dried with 2 LPM nitrogen flow rate (fast drying conditions) (lot 00387-080).

FIG. 2 is a plot of particle size distribution of BSA-loaded microparticles dried with 0.2 LPM nitrogen flow rate (slow drying conditions) (lot 00387-075)

FIG. 3 is a plot of drying profiles of BSA-loaded microparticle showing the change in residual moisture over time in the drying apparatus.

FIG. 4 is a plot of cumulative in vitro release profiles of BSA-loaded microparticles prepared using different rates of drying.

### DETAILED DESCRIPTION

Before the present compounds, compositions, composites, articles, devices, methods, or uses are disclosed and described, it is to be understood that the aspects described below are not limited to specific compounds, compositions, composites, articles, devices, methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

A weight percent of a component, unless specifically stated to the contrary, is based on the total weight of the formulation or composition in which the component is included.

The term “biocompatible” refers a substance that is substantially non-toxic to a subject.

“Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject.

The term “microparticle” is used herein to refer generally to a variety of structures having sizes from about 10 nm to 2000 microns (2 millimeters) and includes microcapsule, microsphere, nanoparticle, nanocapsule, nanosphere as well as particles, in general, that are less than about 2000 microns (2 millimeters). In one aspect, the bioactive agent is encapsulated in the microparticle.

A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. A “releasable bioactive agent” is one that can be released from a disclosed microparticle. Bioactive agents also include those substances which affect the structure or function of a subject, or a pro-drug, which becomes bioactive or more bioactive after it has been placed in a predetermined physiological environment.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

As described above, providing control over the delivery of a bioactive agent can be desirable when traditional formulations are not desirable for use. For example, some therapies may desire the slow release of a bioactive agent, the fast release of a bioactive agent, delivery of the bioactive agent to specific tissues or fluids of a subject, or delivery of two or more agents, among many other delivery modes.

In one aspect, as described herein, methods are provided for processing a microparticle that contains a bioactive agent therein to affect the release profile of the bioactive agent. In general, the release profile can be any desired release profile, depending on the therapy for which the bioactive agent will be used. In a further aspect, the release profile is one or more of controlled-release, extended-release, modified-release, sustained-release, pulsatile-release, delayed-release, or programmed-release, including cyclical-release.

In one aspect, a release profile for a bioactive agent is selected. The selection of the release profile can occur at any time. In various aspects, the selection of the release profile can be made prior to, at, during, or after, the manufacture of the microparticle comprising the bioactive agent. In one aspect, a release profile can be selected prior to the manufacture of the microparticle, and the microparticle constituents can be selected so as to affect the selected release profile. In a further aspect, a release profile can be selected after the manufacture of the microparticle, and the final microparticle processing parameters can be selected and/or adjusted to provide the selected release profile.

In a still further aspect, a release profile can be selected, and the microparticle manufacturing parameters can be iteratively adjusted so as to approach or achieve the selected release profile. The release profile can be achieved or substantially achieved. To that end, a microparticle can be made that provides a release profile that approaches a selected release profile, but that might not necessarily achieve the selected profile. In a further aspect, the selected release profile can be achieved.

Microparticles can be made by any emulsion method known in the art. First, a polymer, a releasable agent, e.g., a bioactive agent, and an organic solvent for the polymer are mixed. An emulsion of this mixture can be formed through the addition of water, which is typically used as the continouous process medium. The emulsion will usually comprise organic droplets having the polymer, the organic solvent, and the releasable agent therein. The organic solvent is then removed by various means, such as liquid extraction, solvent evaporation, etc., which leaves behind a slurry comprising the microparticles having the releasable agent therein. The slurry can also comprise residual water and/or residual organic solvent.

In one aspect, the disclosed methods relate to processing a slurry comprising the microparticle so as to affect the release profile of a bioactive agent contained in or on the microparticle. In general, the slurry can comprise any other suitable material in addition to the microparticle and the bioactive agent, depending on the selected processing conditions. In one aspect, the slurry comprises a liquid or solvent. In a further aspect, the slurry comprises water. In a still further aspect, the slurry comprises at least one organic solvent. In one aspect, the slurry can comprise both aqueous and organic solvents. The microparticle is typically dispersed or suspended in the slurry.

In a further aspect, the slurry comprising the microparticle can be dried under a set of drying parameters such that the selected release profile is substantially achieved. Thus, in one aspect, the way in which a microparticle is dried can affect the release profile of the bioactive agent contained in the microparticle. Without wishing to be bound by theory, it is believed that the way in which a microparticle is dried may affect the morphology of the microparticle, which thereby affects the release profile of the bioactive agent contained therein.

In one aspect, the drying parameters which enable the selected release profile to be achieved can be known or unknown prior to drying the microparticle to achieve the selected release profile. Thus, in one aspect, in order to substantially achieve the selected release profile, multiple microparticle drying runs can be useful. For example, a microparticle can be formulated and dried under a set of drying parameters, and the release profile of the bioactive agent contained in the microparticle can be recorded. If the release profile is not substantially close to the selected or desired release profile, the drying parameters can be adjusted, iteratively, until the selected or desired release profile is substantially achieved. By contrast, it may be possible to predict which set of drying parameters will provide the selected release profile.

In general, the drying parameters can be any drying parameter. For air drying using non-mechanical means, for example, the drying parameters can be one or more of temperature, pressure, humidity, or drying time. In a further aspect, for mechanical drying, the drying parameters can be one or more of temperature, pressure, humidity, drying time, stirring speed, agitation speed, or gas-flow on, in, or through the slurry. The gas flow-rate can range, for example, from 0.2 to about 2.0 L/min. The drying gas can be any inert gas, such as air, nitrogen, argon, and the like.

The microparticles can be dried under a set of drying using any suitable method. When a dryer is used with the methods disclosed herein, the dryer can be any dryer which substantially achieves the results intended herein. In one aspect, the microparticle can be dried using an agitated filter dryer. In a further aspect, a disclosed method comprises drying a microparticle having a bioactive agent therein in an agitated filter dryer. An agitated filter dryer is a drying device known in the art. Typically, an agitated filter dryer comprises a vessel with a filter plate at the bottom of the vessel. A slurry can be loaded into the vessel, and dried optionally under reduced pressure (e.g., vacuum) and/or under a stream of gas, such as air or an inert gas, such as nitrogen or argon, with mechanical agitation of the slurry by scraping blades, which are driven by an agitator shaft. The entire vessel can be kept at desired temperature by having a jacketed vessel, and/or other jacketed components through which heat transfer medium can be passed. Drying parameters for an agitated filter dryer can include without limitation, temperature, pressure, humidity, drying time, agitation speed, amount of washing medium, including the number of washings, speed of scraping blades, amount of venting, discharge mechanism, inert gas in-flow, among others. In general, any type of agitated filter dryer can be used. In a further aspect, a Nutsche filter can be used. A Nutsche filter is a type of agitated filter dryer. In a still further aspect, a stirred cell dryer can be used, such as a commercially available stirred cell dryer, e.g., a MILLIPORE stirred cell dryer.

It should be appreciated that in one aspect, the use of an agitated filter dryer can be beneficial to overcome challenges encountered with traditional bulk material drying methods in the microparticle art, including, for example, the use of Sweco dryers. A Sweco dryer is typically comprised of a vessel which shakes to agitate the bulk material. Oftentimes with the use of a Sweco dryer, large agglomerates can form, which have to be separated from the final powder. This is partly due to the fact that traditional Sweco dryers are not equipped with a mechanical stirring or scraping means, but rather simply shake a vessel of bulk material while drying. It should be appreciated that, in one aspect, the use of an agitated filter dryer can provide for good particle distrubution with fewer agglomerates, and also allow for the control of the drying parameters, as discussed above. In a further aspect, however, a microparticle can be dried using a Sweco dryer.

In one aspect, microparticles are disclosed that are produced by the disclosed methods. In general, the microparticles disclosed herein can be any suitable microparticle. In one aspect, a bioactive agent is encapsulated within the microparticle. In another aspect, the bioactive agent is associated with the microparticle. In one aspect, the microparticle comprises a suitable biocompatible and biodegradable or non-biodegradable polymer. The polymers can be homopolymers or copolymers, including without limitation block or blocky co- or ter-polymers, random co- or ter-polymers, star polymers, or dendrimers. Any desired molecular weight polymer can be used, depending on the desired properties of the microparticle. In certain aspects, if a high strength microparticle is desired, then high molecular weight polymers can be used, for example, to meet strength requirements. In other aspects, low or medium molecular weight polymers can be used when, for example, when resorption time of the polymer, rather than material strength is desired.

When a biodegradable polymer is used, the microparticle can be formulated so as to degrade within a desired time interval, once present in a subject. In some aspects, the time interval can be from about less than one day to about 1 month. Longer time intervals can extend to 6 months, including for example, polymer matrices that degrade from about ≧0 to about 6 months, or from about 1 to about 6 months. In other aspects, the polymer can degrade in longer time intervals, up to 2 years or longer, including, for example, from about ≧0 to about 2 years, or from about 1 month to about 2 years.

In one aspect, the desired release profile can influence the selection of the polymer. A biocompatible polymer, for example, can be selected so as to release or allow the release of a bioactive agent therefrom at a desired lapsed time after the microparticle has been administered to a subject. For example, the polymer can be selected to release or allow the release of the bioactive agent prior to the bioactive agent beginning to diminish its activity, as the bioactive agent begins to diminish in activity, when the bioactive agent is partially diminished in activity, for example at least 25%, at least 50% or at least 75% diminished, when the bioactive agent is substantially diminished in activity, or when the bioactive agent is completely gone or no longer has activity.

In one aspect, the polymer can be one or more of polyesters, polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

In a further aspect, the polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

In a still further aspect, useful biocompatible polymers are those that comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In a still further aspect, useful biocompatible polymers are those that comprise one or more residues of lactide, glycolide, caprolactone, or a combination thereof.

In one aspect, useful biodegradable polymers are those that comprise one or more blocks of hydrophilic or water soluble polymers, including, but not limited to, polyethylene glycol, (PEG), or polyvinyl pyrrolidone (PVP), in combination with one or more blocks another biocompabible or biodegradable polymer that comprises lactide, glycolide, caprolactone, or a combination thereof.

In specific aspects, the biodegradable polymer can comprise one or more lactide residues. To that end, the polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL- polymers. In addition to copolymers comprising glycolide and DL-lactide or L-lactide, copolymers of L-lactide and DL-lactide are commercially available. Homopolymers of lactide or glycolide are also commercially available.

When the biodegradable polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

In a further aspect, the polymer can be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co- caprolactone), 65:35 poly(lactide-co- caprolactone), or 50:50 poly(lactide-co- caprolactone), where the ratios are mole ratios.

It is understood that any combination of the aforementioned biodegradable polymers can be used, including, but not limited to, copolymers thereof, mixtures thereof, or blends thereof. Likewise, it is understood that when a residue of a biodegradable polymer is disclosed, any suitable polymer, copolymer, mixture, or blend, that comprises the disclosed residue, is also considered disclosed. To that end, when multiple residues are individually disclosed (i.e., not in combination with another), it is understood that any combination of the individual residues can be used.

In general, the microparticle can be any microparticle made from a suitable starting material. In one aspect, as described above, the microparticle can be made out of a suitable polymer. The microparticle can contain and effect the release of the bioactive agent contained therein. The microparticle can be comprised of any of those polymers mentioned above or any polymer used in the microparticle art. In general, the above mentioned polymers can be cross-linked to a certain level, which thereby can form a microparticle of the polymer, as is known in the art.

In one aspect, the disclosed microparticles can have an average or mean particle size of from about 20 microns to about 125 microns. In one embodiment the range of mean particle size is from about 40 microns to about 90 microns. In another embodiment the range of mean particle sizes is from about 50 microns to about 80 microns. Particle size distributions are measured by laser diffraction techniques known to those of skill in the art.

In a further aspect, the bioactive agent can be encapsulated, microencapsulated, or otherwise contained within a microparticle. The microparticle can modulate the release of the bioactive agent. The microparticle can comprise any desired amount of the bioactive agent. For example, the microparticle can comprise 1%, 5%, 10%, 20%, 30%, 40%, 50%, 60%, 70%, 80%, 90%, 95% by weight bioactive agent, relative to the weight of the microparticle, including any range between the disclosed percentages.

The microparticles can be made using methods known in the art, including, for example, those methods disclosed in U.S. Patent Publication No. 2007/0190154 to Zeigerson, published Aug. 16, 2007, and U.S. Pat. No. 5,407,609 to Tice et al., both of which are incorporated herein in their entirety by this reference for teachings of microparticle preparation methods. As will be apparent, depending upon processing conditions, the polymer used as a starting material in the admixing step may or may not be the same polymer present in the final implantable composite. For example, the polymer during processing may undergo polymerization or depolymerization reactions, which ultimately can produce a different polymer that was used prior to processing. Thus, the term “polymer” as used herein covers the polymers used as starting materials as well as the final polymer present in the device produced by the methods described herein. Methods for making microparticles can be used in combination with the drying methods and dyring parameters described above.

As discussed above, the microparticle comprises a bioactive agent. The bioactive agent can be a releasable bioactive agent, i.e., a bioactive agent that can be released from the microparticle into adjacent tissues or fluids of a subject. In certain aspects, the bioactive agent can be in or on the microparticle.

Various forms of the bioactive agent can be used, which are capable of being released from the microparticle into adjacent tissues or fluids. To that end, a liquid or solid bioactive agent can be incorporated into the implantable composites described herein. The bioactive agents are at least very slightly water soluble, and preferably moderately water soluble. The bioactive agents can include salts of the active ingredient. As such, the bioactive agents can be acidic, basic, or amphoteric salts. They can be nonionic molecules, polar molecules, or molecular complexes capable of hydrogen bonding. The bioactive agent can be included in the compositions in the form of, for example, an uncharged molecule, a molecular complex, a salt, an ether, an ester, an amide, polymer drug conjugate, or other form to provide the effective biological or physiological activity.

Examples of bioactive agents that incorporated into systems herein include, but are not limited to, peptides, proteins such as hormones, enzymes, antibodies and the like, nucleic acids such as aptamers, iRNA, DNA, RNA, antisense nucleic acid or the like, antisense nucleic acid analogs or the like, low-molecular weight compounds, or high-molecular-weight compounds. Bioactive agents contemplated for use in the disclosed implantable composites include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents in the microparticles include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

The microparticle can comprise a large number of bioactive agents either singly or in combination. Other bioactive agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenytoin sodium, diazepam, and the like; antidepressants such as isocarboxazid, amoxapine, and the like;antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B₁₂, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, or a combination thereof. In one aspect, the bioactive agent can be a combination of Rifampicin (Rifampin in U.S.) and Minocycline.

In certain aspects, the bioactive agent can be present as a component in a pharmaceutical composition. Pharmaceutical compositions can be conveniently prepared in a desired dosage form, including, for example, a unit dosage form or controlled release dosage form, and prepared by any of the methods well known in the art of pharmacy. In general, pharmaceutical compositions are prepared by uniformly and intimately bringing the bioactive agent into association with a liquid carrier or a finely divided solid carrier, or both. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other pharmaceutically acceptable carriers or components that can be mixed with the bioactive agent can include, for example, a fatty acid, a sugar, a salt, a water-soluble polymer such as polyethylene glycol, a protein, polysacharride, or carboxmethyl cellulose, a surfactant, a plasticizer, a high- or low-molecular-weight porosigen such as polymer or a salt or sugar, or a hydrophobic low-molecular-weight compound such as cholesterol or a wax.

The microparticle can be administered to any desired subject. The subject can be a vertebrate, such as a mammal, a fish, a bird, a reptile, or an amphibian. The subject of the herein disclosed methods can be, for example, a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Microsphere Production/Drying

Bovine serum albumin, BSA, (Fraction V, Sigma Chemical Co., St. Louis, Mo.) was added to a 20% solution of 5050 PLG 4.5E polymer (Lakeshore Biomaterials brand polymer from SurModics Pharmaceuticals, Birmingham, Ala.) in ethyl acetate at a level of 10% based on the combined weight of polymer and BSA. BSA was suspended in the polymer solution using an IKA Ultraturrax at 13,500 rpm for 30 seconds. The drug/polymer suspension (15 ml) was then emulsified at a rate of 15 mL/min into an aqueous solution containing 2 wt % poly(vinyl alcohol) (PVA) that was fed at a rate of 150 mL/min by using a Silverson L4RT-A homogenizer at 860 rpm. The resulting o/w emulsion was then extracted into deionized water which was fed at a rate of 1450 mL/min and stirred an additional 60 minutes to extract the organic solvent and form polymer microparticles. The resulting microsphere suspension was passed through 150 and 25 micron test sieves (Retsch GmbH) in order to isolate the microparticle fraction between 25-250 microns in size. The product collected on the 25 micron sieve was washed with deionized water (1 liter) and was then transferred to a drying apparatus.

The drying apparatus was a modified MILLIPORE stirred cell (MILLIPORE Stirred Cell 8200, Fisher Scientific) where a 25 μm sieve mesh material (Retsch GmbH) was used as the bottom filter membrane. This drying apparatus was then connected to a nitrogen source. Nitrogen flow was initiated to remove excess water from the system to form a solid microsphere cake at the bottom of the cell. Nitrogen flow was then controlled through the cell using a regulator Drying conditions were selected that allowed for either fast or slow drying conditions based on the nitrogen flow rate through the drying apparatus. Fast drying conditions used a nitrogen flow rate of 2 LPM while slow drying conditions used a flow rate of 0.2 LPM. Aliquots of microspheres were taken throughout the course of drying and the microsphere cake was stirred at each time point to promote even drying throughout the sample. Once the product was dry, it was collected and reserved for further analysis.

Particle Size Analysis

Particle size analysis was performed by laser diffraction using a Beckman Coulter LS13,320 particle size analyzer. The fraunhoffer model was used to compute size distribution based on volume-averaged statistics. Particles sizes were conducted on portions of the bulk microparticle product taken just prior to collection on the test sieves. Reported particle size results are in microns and include the mean size (mean), and the particle sizes at 10% and 90% of the particle size distribution (D10 and D90, respectively).

Residual Moisture and Residual Solvent Analysis

Samples pulled throughout the course of drying were analyzed for residual moisture content by a Computrac Vapor Pro moisture analyzer (Arizona Instruments). Residual solvent analysis for ethyl acetate was performed by gas chromotography (GC).

BSA Content

The final product was analyzed for BSA content by accurately weighing 10 mg of microspheres into a test tube and adding 3 mL of ethyl acetate. This mixture was vortexed for 30 seconds, then centrifuged at 3500 rpm for 15 minutes. Supernatant was removed, 3 mL of ethyl acetate added, mixture vortexed for 30 seconds, and centrifuged at 3500 rpm for 15 minutes. This procedure was repeated one last time for a total of three ethyl acetate washes. After the last wash and removal of supernatant, the remaining BSA pellet was dried under nitrogen flow to remove excess ethyl acetate. Next, 3 mL of phosphate buffered saline (PBS, 1×) (Fisher Scientific) was added to the dried BSA material and the mixture was vortexed for 30 seconds to completely dissolve the BSA. This mixture was then centrifuged for 15 minutes at 3500 rpm to remove any residual polymer precipitate. The supernatant containing the water-soluble BSA was then collected and analyzed by HPLC for BSA content.

In-Vitro Release

In vitro studies were performed by accurately weighing 30 mg of BSA loaded microspheres into a test tube, adding 3 mL of 1× PBS, and placing samples in a 37° C. static incubator. At each time point a serum separator was used to filter the microspheres from the release buffer, and the release buffer was collected for analysis. At each time point, the total volume of release buffer was removed and fully replaced with fresh 1× PBS. Each lot of microspheres was evaluated in triplicate for the release study. Samples were analyzed by HPLC.

HPLC Method

HPLC analysis was performed on a Perkin Elmer instrument using the following parameters: Shodex Protein KW-803 column, 214 nm detection wavelength, 1 mL/min mobile phase flow rate, 10 μL sample injection volume, and 20 minute run time per sample. Sample tray and column were kept at ambient temperature. Mobile phase was a pre-mixed 1:1 100 mM Sodium Phosphate:100mM Sodium Sulfate, pH 7.

Results

Properties of the following samples were evaluated:

TABLE 1
Samples.

| | | Nitrogen flow rate during |
|---|---|---|
| Batch ID | Comment | drying (LPM) |
| 00387-080 | Fast drying conditions | 2 |
| 00387-075 | Slow drying conditions | 0.2 |

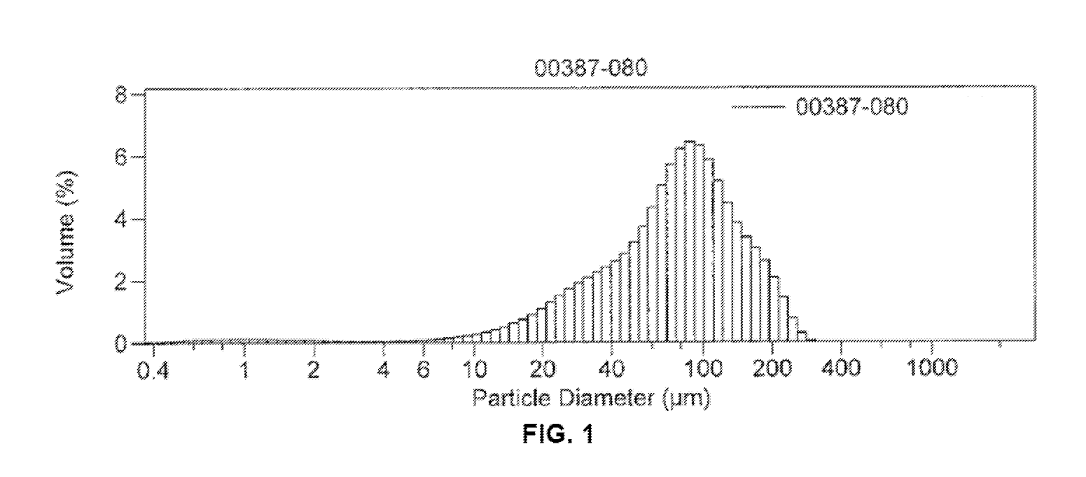

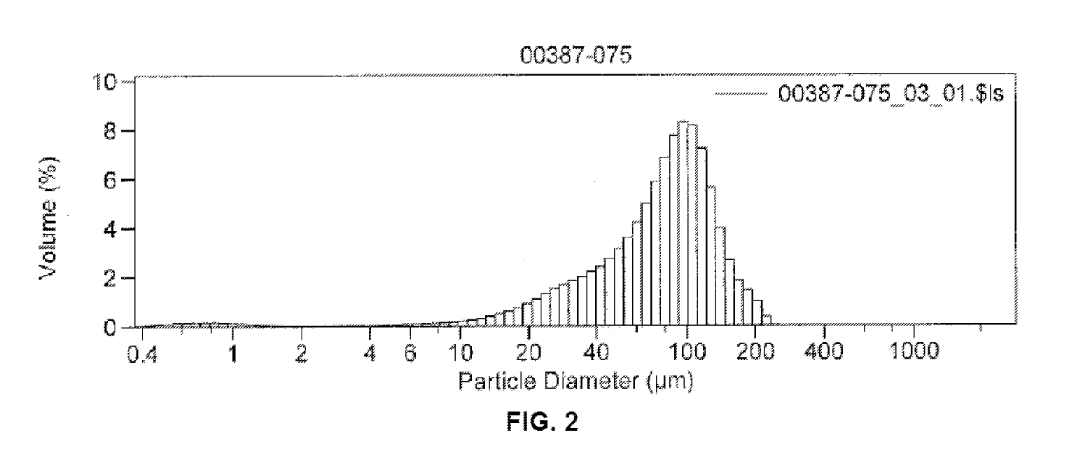

Particle sizes of the samples from Table 1 are listed below in Table 2. Particle size distributions for these samples are shown in FIGS. 1 and 2.

TABLE 2
Particle Size.

| | Drying Rate | D10, | Mean, | D90, |
|---|---|---|---|---|
| Batch ID | (LPM) | microns | microns | microns |
| 00387-080 | 2 | 24.24 | 86.22 | 162.3 |
| 00387-075 | 0.2 | 26.73 | 82.79 | 137.8 |

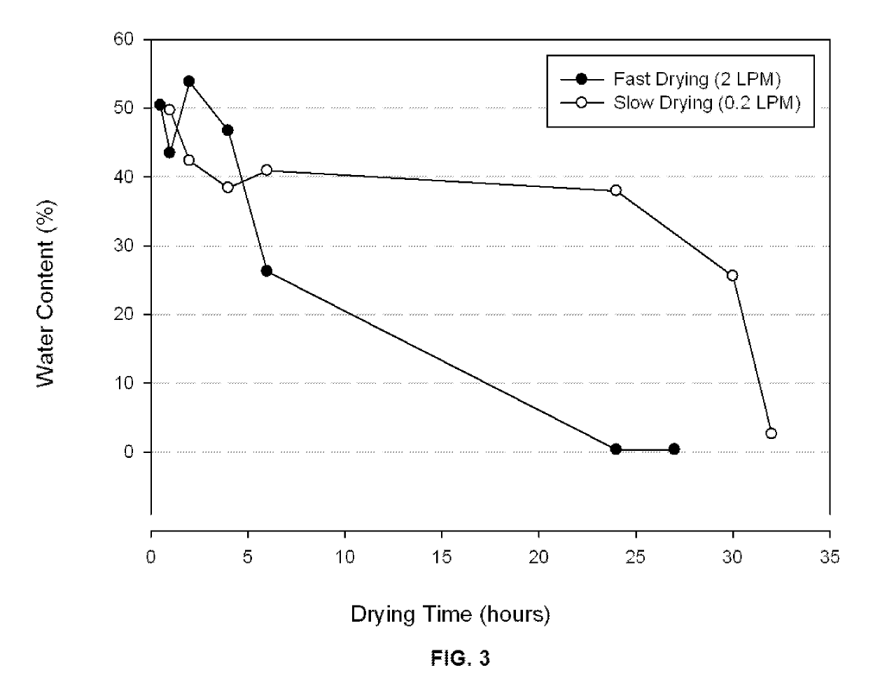

Drying profiles of the two samples discussed above are shown in FIG. 3. As can be seen from FIG. 3, faster drying (i.e., with a higher nitrogen flow-rate) reduces water content faster than slower drying. Table 3 lists residual solvent for each of the samples. Drug loading after drying is shown in Table 4.

TABLE 3
Residual Solvent.

| | Drying Rate | Residual Solvent—Ethyl |
|---|---|---|
| Batch ID | (LPM) | Acetate |
| 00387-080 | 2 | 4.8% |
| 00387-075 | 0.2 | 0.9% |

TABLE 4
BSA Content after drying.

| | Drying Rate | |
|---|---|---|
| Batch ID | (LPM) | Total Core Loading (%) |
| 00387-080 | 2 | 8.69 |
| 00387-075 | 0.2 | 8.39 |

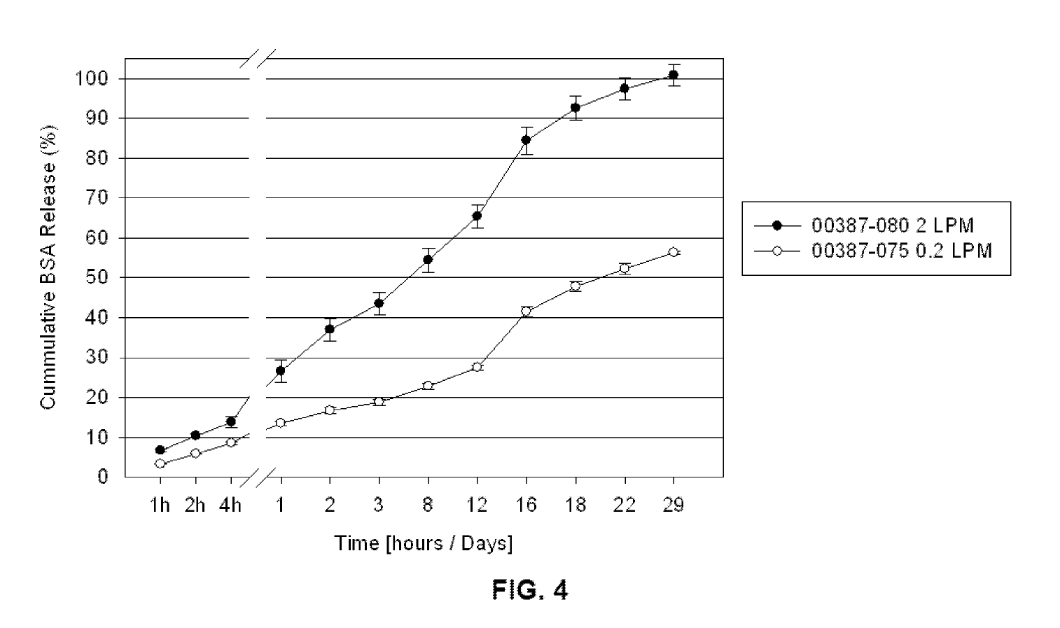

With reference to FIG. 4, which shows the cumulative release profile for the two samples, demonstrates that by tuning the drying profile, a different release profile can be achieved. Specifically, in this case, slower drying led to a slower rate of release relative to the microspheres dried under fast conditions.

Various modifications and variations can be made to the compounds, composites, kits, articles, devices, compositions, and methods described herein. Other aspects of the the compounds, composites, kits, articles, devices, compositions, and methods described herein will be apparent from consideration of the specification and practice of the the compounds, composites, kits, articles, devices, compositions, and methods disclosed herein. It is intended that the specification and examples be considered as exemplary.

1. A method for preparing a microparticle having a selected release profile for the release of a bioactive agent contained therein, the method comprising:
a) providing a slurry comprising a microparticle having a releasable bioactive agent therein;
b) selecting a release profile for the bioactive agent;
c) selecting a set of drying parameters to achieve a drying rate such that the selected release profile is substantially achieved; and
d) drying the microparticle under a set of drying parameters
wherein the rate of drying is directly proportional with the release rate of the bioactive agent from the microparticle; and wherein a fast drying rate is chosen to achieve a fast release rate and a slow drying rate is chosen to achieve a slow release rate.

2. The method of claim 1, wherein the microparticle is dried using an agitated filter dryer.

3. The method of claim 1, wherein the microparticle is dried using a Nutsche filter dryer.

4. The method of claim 1, wherein the slurry comprises water.

5. The method of claim 1, wherein the slurry comprises at least one organic.

6. The method of claim 1, wherein the slurry comprises ethyl acetate.

7. The method of claim 1, wherein the microparticle comprises poly(lactide), poly(glycolide), poly(caprolactone), poly(lactide-co-glycolide), or a combination thereof.

8. The method of claim 1, wherein the microparticle is dried using a stirred cell filter dryer.

9. The method of claim 8, wherein the microparticle is dried under gas at a gas flow rate ranging from 0.2 to 2 liters per minute.

10. A microparticle made by the method of claim 1.

11. A method for drying a microparticle, comprising:
a) providing a slurry comprising a microparticle having a releasable bioactive agent therein; and
b) drying the microparticle using an agitated filter dryer or a stirred cell filter dryer under a set of selected drying parameters selected to achieve a drying rate;
wherein the rate of drying is directly proportional with the release rate of the bioactive agent from the microparticle; and wherein a fast drying rate is chosen to achieve a fast release rate and a slow drying rate is chosen to achieve a slow release rate.

12. The method of claim 11, wherein the microparticle is dried using a Nutsche filter dryer.

13. The method of claim 11, wherein the slurry comprises water.

14. The method of claim 11, wherein the slurry comprises at least one organic.

15. The method of claim 11, wherein the slurry comprises ethyl acetate.

16. The method of claim 11, wherein the microparticle comprises poly(lactide), poly(glycolide), poly(caprolactone), poly(lactide-co-glycolide), or a combination thereof.

17. The method of claim 11, wherein the microparticle is dried under gas at a gas flow rate ranging from 0.2 to 2 liters per minute.

18. A microparticle made by the method of claim 11.
